SEQUENCE LISTING (39 sequences)

SEQ ID NO: 1 - DNA, 23 bp, synthetic construct
  source 1..23: mol_type = other DNA
tgaagtttttgacttaataaatg

SEQ ID NO: 2 - DNA, 23 bp, synthetic construct
  source 1..23: mol_type = other DNA
gagggcaggtctttttctggacc

SEQ ID NO: 3 - DNA, 23 bp, synthetic construct
  source 1..23: mol_type = other DNA
ggcctgaggagtagcagaagctg

SEQ ID NO: 4 - DNA, 23 bp, synthetic construct
  source 1..23: mol_type = other DNA
cttacatgtcttatctcacctga

SEQ ID NO: 5 - DNA, 23 bp, synthetic construct
  source 1..23: mol_type = other DNA
ggttctatttggcttgtatgaag

SEQ ID NO: 6 - DNA, 23 bp, synthetic construct
  source 1..23: mol_type = other DNA
taaaaaagtcttttccaagactt

SEQ ID NO: 7 - DNA, 23 bp, synthetic construct
  source 1..23: mol_type = other DNA
taaagtctctgacttaacaactt

SEQ ID NO: 8 - DNA, 23 bp, synthetic construct
  source 1..23: mol_type = other DNA
ctggctcataaattaacccttga

SEQ ID NO: 9 - DNA, 23 bp, synthetic construct
  source 1..23: mol_type = other DNA
gttgctatttttatttccttctc

SEQ ID NO: 10 - DNA, 23 bp, synthetic construct
  source 1..23: mol_type = other DNA
tcaggaaaacagtaatgggaaca

SEQ ID NO: 11 - DNA, 23 bp, synthetic construct
  source 1..23: mol_type = other DNA
ttatacatttcattctaacgtgt

SEQ ID NO: 12 - DNA, 23 bp, synthetic construct
  source 1..23: mol_type = other DNA
cttggaaaaatagaaaataaata

SEQ ID NO: 13 - DNA, 23 bp, synthetic construct
  source 1..23: mol_type = other DNA
tgttatgtatgcatttttattat

SEQ ID NO: 14 - DNA, 23 bp, synthetic construct
  source 1..23: mol_type = other DNA
cgggcttagccgagacctatcac

SEQ ID NO: 15 - DNA, 23 bp, synthetic construct
  source 1..23: mol_type = other DNA
cagaacttgttgggagtttacgg

SEQ ID NO: 16 - DNA, 23 bp, synthetic construct
  source 1..23: mol_type = other DNA
ggaagcacgtcacacagccgagg

SEQ ID NO: 17 - DNA, 23 bp, synthetic construct
  source 1..23: mol_type = other DNA
ggtctatccttcaaggagaggaa

SEQ ID NO: 18 - DNA, 23 bp, synthetic construct
  source 1..23: mol_type = other DNA
tccgtgaagctcggtgtccataa

SEQ ID NO: 19 - DNA, 23 bp, synthetic construct
  source 1..23: mol_type = other DNA
gcagcctgtttgtaatcccagca

SEQ ID NO: 20 - DNA, 23 bp, synthetic construct
  source 1..23: mol_type = other DNA
aataaagggcacggtgcgtgcgc

SEQ ID NO: 21 - DNA, 20 bp, synthetic construct
  source 1..20: mol_type = other DNA
tctcccggatcttgtatata

SEQ ID NO: 22 - DNA, 20 bp, synthetic construct
  source 1..20: mol_type = other DNA
catgtcttatctcacctact

SEQ ID NO: 23 - DNA, 20 bp, synthetic construct
  source 1..20: mol_type = other DNA
agggtaagatcactatcaga

SEQ ID NO: 24 - DNA, 20 bp, synthetic construct
  source 1..20: mol_type = other DNA
tctttgctttacccacagaa

SEQ ID NO: 25 - DNA, 20 bp, synthetic construct
  source 1..20: mol_type = other DNA
ctggttctgtgggtaaagca

SEQ ID NO: 26 - DNA, 20 bp, synthetic construct
  source 1..20: mol_type = other DNA
gggctgtcaggtttgctggt

SEQ ID NO: 27 - DNA, 20 bp, synthetic construct
  source 1..20: mol_type = other DNA
ttcattcacttcattcattc

SEQ ID NO: 28 - DNA, 20 bp, synthetic construct
  source 1..20: mol_type = other DNA
acaagcatcagtttagggct

SEQ ID NO: 29 - DNA, 20 bp, synthetic construct
  source 1..20: mol_type = other DNA
tttggcttatacgtagaaaa

SEQ ID NO: 30 - DNA, 20 bp, synthetic construct
  source 1..20: mol_type = other DNA
ggaatctcctacctgccccc

SEQ ID NO: 31 - DNA, 20 bp, synthetic construct
  source 1..20: mol_type = other DNA
ttatagtgaaagtttccccc

SEQ ID NO: 32 - DNA, 20 bp, synthetic construct
  source 1..20: mol_type = other DNA
aggcccctagtggtttaatc

SEQ ID NO: 33 - DNA, 20 bp, synthetic construct
  source 1..20: mol_type = other DNA
cgcggcagtaaagattaaac

SEQ ID NO: 34 - DNA, 20 bp, synthetic construct
  source 1..20: mol_type = other DNA
cacatttcattgctcgtgtg

SEQ ID NO: 35 - DNA, 20 bp, synthetic construct
  source 1..20: mol_type = other DNA
gtcaacctctcaagcttgga

SEQ ID NO: 36 - DNA, 20 bp, synthetic construct
  source 1..20: mol_type = other DNA
tgtgtgcatattggaaaaca

SEQ ID NO: 37 - DNA, 20 bp, synthetic construct
  source 1..20: mol_type = other DNA
tattacaagtagtgtttgcg

SEQ ID NO: 38 - DNA, 20 bp, synthetic construct
  source 1..20: mol_type = other DNA
cattatttctctagtgtcgt

SEQ ID NO: 39 - DNA, 20 bp, synthetic construct
  source 1..20: mol_type = other DNA
cccctagttccaaccgtata

(19) United States
(12) Patent Application Publication
(10) Pub. No.: US 2026/0108628 A1
(43) Pub. Date: Apr. 23, 2026

(54) SGRNA FOR UTROPHIN ACTIVATION TO TREAT DUCHENNE MUSCULAR DYSTROPHY AND USE THEREOF

(71) Applicant: KUNMING UNIVERSITY OF SCIENCE AND TECHNOLOGY, Kunming (CN)

(72) Inventors: Yongchang CHEN, Kunming (CN); Ruo WU, Kunming (CN); Peng LI, Kunming (CN); Wenting GUO, Kunming (CN)

(73) Assignee: KUNMING UNIVERSITY OF SCIENCE AND TECHNOLOGY, Kunming (CN)

(21) Appl. No.: 19/144,537

(22) PCT Filed: Jan. 18, 2024
(86) PCT No.: PCT/CN2024/073030

§ 371 Date: Jun. 30, 2025

(30) Foreign Application Priority Data
Jan. 18, 2023 (CN) 202310084593.3

(51) Int. Cl. A61K 48/00; A61K 31/7105; C12N 15/113; C12N 15/864

(52) U.S. Cl. CPC A61K 48/005; A61K 31/7105; C12N 15/113; C12N 15/8645; C12N 2750/14143; C12N 2800/107

(57) ABSTRACT

An sgRNA for treating Duchenne muscular dystrophy by the activation of utrophin, and a use are provided. The sgRNA is an sgRNA capable of activating the transcription and translation expression of human or mouse utrophin; and the DNA sequence of the sgRNA is as represented by SEQ ID NOS: 1-20 or SEQ ID NOS: 21-39. By means of sgRNAs targeting endogenous utrophin, the sgRNAs can efficiently activate the transcription and translation expression of human and mouse utrophin, thus providing a therapeutic option for the treatment of Duchenne muscular dystrophy.

### CROSS REFERENCE TO THE RELATED APPLICATIONS

This application is the national phase entry of International Application No. PCT/CN2024/073030, filed on Jan. 18, 2024, which is based upon and claims priority to Chinese Patent Application No. 202310084593.3, filed on Jan. 18, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBHNZH003-PKG_SequenceListing.xml, created on 07/08/2025, and is 35,508 bytes in size.

### TECHNICAL FIELD

The present invention relates to the technical field of biology, and in particular, relates to a single-guide RNA (sgRNA) for utrophin activation to treat Duchenne muscular dystrophy and use thereof.

### BACKGROUND

Duchenne muscular dystrophy (DMD) is a severe progressive muscle-wasting disease. The earliest symptoms include difficulty in climbing stairs, difficulty in standing up, and frequent falling; and patients develop these symptoms around the age of 2-3. Most patients begin to rely on wheelchairs around the age of 10-12 and need assisted ventilation around the age of 20. With optimal care, most DMD patients die of heart and respiratory failures between the age of 20 and 40. DMD is a hereditary muscular dystrophy caused by the loss of dystrophin due to a mutation in the DMD gene. The DMD gene occurs in Xp21.1 to p21.2(31119218 to 33339608), with a total length of 2220391 bp, including 79 exons and 7 promoters, and its coding region has a total length of 14 kb, making it one of the largest genes known at present. According to statistical data from the website www.umd.be/TREAT_DMD/, thousands of mutations in the human DMD gene have now been reported, and deletions may occur almost anywhere in this gene. These limiting factors lead to the necessity of personalized treatment for DMD gene repair. UTRN gene is ubiquitous on 6q24 in human autosome 6. Its coding homology and protein structure characteristics are 80% similar to those of dystrophin. Despite their highly homogeneous and relatively conservative structures, utrophin and dystrophin exhibit different spatiotemporal expressions. Utrophin is localized at the tendon and neuromuscular junction in the normal human muscular system, and there is no report of diseases induced by utrophin mutations. In the muscles of DMD patients, utrophin has an upregulated expression due to a special compensatory mechanism, and in some cases, it is also localized on the muscle cell membrane. As the disease progresses, myofibers undergo an alternating process between inflammatory necrosis and neogenesis until the muscle stem cell bank is exhausted. In the muscles of DMD patients, most of the muscle tissues are replaced by connective tissues and adipose tissues. The expression of utrophin is mainly localized on new myofibers, and the expression of utrophin changes periodically with the progression of the disease and finally returns to a lower level in case of very serious amyotrophy.

Since the utrophin was first reported in 1989, numerous studies have gradually emerged on whether utrophin can be an alternative for dystrophin treatment. Not until 2008 did Odom et al. clearly define the therapeutic effect of utrophin by delivering an exogenously synthesized mini-version of utrophin, that retained the key functional domains of utrophin, into mdx mice and utrophin/dystrophin-deficient (DKO) mice to successfully alleviate the disease state of mice. Since then, a large number of utrophin-based endogenous activation experiments and exogenous replenishment methods have been constantly carried out at the preclinical animal and cellular levels. Utrophin overexpression can compensate for dystrophin function losses in mdx mice and GRMD dogs. It is essential for utrophin to distribute and localize along the muscle membrane, and a 2-fold increase in low dose has been proved to be beneficial. Utrophin at high level can also rescue serological markers such as CK and ameliorate muscle inflammation pathology and function in a dose-dependent manner. In addition, the inflammatory factors detected in dogs show that, unlike the micro-dystrophin therapy, immune responses induced by utrophin are barely observed. Current therapies for utrophin consist mainly in the following aspects.

### (1) Pharmacological Utrophin Activator

Transcriptional regulation is carried out on the promoter of utrophin by using orally bioavailable small molecules to provide a systemic solution targeting skeletal and cardiac muscles for all DMD patients, regardless of dystrophin mutations. In vitro screening is carried out by using mdx mouse's immortalized H2K-mdx cell, which has an 8.9-kb fragment of the human utrophin promoter attached to a luciferase reporter gene, resulting in the discovery of a first orally bioavailable small molecule utrophin up-regulator: ezutromid (officially SMT C1100), with an active ingredient 5 (ethylsulfonyl)-2-(naphth-2-yl) benzoxazole formulated for oral administration. In human cells, it has been demonstrated that the in vitro upregulation of utrophin increases by 25% and 50% at the mRNA and protein levels, respectively, in human cells inoculated with ezutromid. In mdx animals, daily oral administration of ezutromid increases the expression of utrophin in the skeletal muscle, respiratory tract, and cardiac muscle to thus improve myolemma stability. Utrophin overexpression leads to a significant decrease in key features (e.g., regeneration, necrosis, and fibrosis) of the DMD disease, leading to the translation into improved physiological and muscular functions. Importantly, ezutromid treatment has been proved to be effective in exercising mdx mice and also effective in combination with the approved gold standard steroid prednisolone. At present, the study has entered the clinical stage. However, further study is also needed due to the poor oral absorption and utilization of a human body.

### (2) Utrophin Gene Therapy

A vector carrying normal utrophin cDNA is used in place of mutant dystrophin to fulfill the dystrophy function in vivo. Early studies on mdx mouse models demonstrated the virus-carried truncated cDNA with the main functional region of UTRN restored the expression of utrophin on the myolemma and improved the muscle function. In particular, the delivery of micro-utrophin by an improved adeno-associated virus (AAV)9 vector restored protein expression and muscle strength in a dog model with symptoms more similar to those of humans. This better demonstrates the feasibility of a gene replacement therapy.

### (3) Utrophin Protein Replacement Therapy

Chimeric proteins encoding the protein transduction domain of the HIV-1 TAT protein fused to full-length TAT-Utr and truncated TAT-μUtr are used for direct replacement of proteins, which can be effectively transduced into the skeletal muscle, cardiac muscle, brain and liver. Cell-penetrating recombinant TAT-μUtr expressed with a baculovirus system can restore the membrane structure, stabilize the myolemma integrity, and mitigate the pathophysiological symptoms in DMD and dko mice. Despite the current encouraging preclinical experimental results, there is no further report on follow-up efficacy.

### (4) Regulation of Utrophin Expression by Micro-RNA

3′UTR in the UTRN gene contains some micro-RNAs (let-7c, miR-150, miR-196b, miR296-5p, and miR-133b) with repressive functions. Mishra and colleagues reported a 2- to 3-fold increase in utrophin expression in the mdx skeletal muscle based on the disruption of the interaction site of Let-7c on utrophin 3′UTR by using 2′-o-methyl oligonucleotide. This is associated with histological benefits and improved muscle strength. Importantly, another recent publication indicated that miR-206 was an important factor capable of stimulating skeletal muscle regeneration, and the overexpression of microRNA led to an increase in utrophin level. As a result, the vesicle transport of dystrophin-glycoprotein and integrin attachment complexes is selectively enhanced, which stabilizes myolemma proteins (e.g., troponin).

At present, the activation mode for utrophin has achieved a certain early treatment effect mainly in animal models such as mice and dogs, but there is no significant difference in the final statistical results between the drug treatment group and the placebo group after the entrance into a clinical trial. Accordingly, there is still a long way to go in terms of the utrophin activation treatment.

Since the reporting of the CRISPR gene editing system, other related systems have been developed based on this system, such as the BE system and the transcriptional activation and transcriptional inhibition systems. By point mutation of the nuclease site of a Cas protein, the Cas protein loses its nuclease activity and thus fails to cleave a target site, but retaining its ability to bind DNA. In this case, the Cas protein turns into dead-Cas. The transcriptional activation system is to activate a target gene by an inactivated Cas protein-coupled activating element under the guidance of sgRNA. However, most of the Cas protein-coupled transcriptional activating elements are larger than 4.7 kb, which far exceeds the loading capacity of an adeno-associated virus (AAC), and splitting is required for delivery. In this way, the effectiveness of the activation system is greatly reduced.

### SUMMARY

In view of the above technical problems that need to be solved, the present invention provides a sgRNA for utrophin activation to treat DMD, and the sgRNA is designed to target endogenous utrophin to provide an alternative therapeutic method for treating DMD. At the same time, sgRNAs with the optimal activation efficiency are screened out at a cellular level and an adult mouse level. These sgRNAs can efficiently activate the transcriptional and translational expressions of human and mouse utrophin.

To achieve the object of the present invention, the present invention provides a sgRNA for utrophin activation to treat Duchenne muscular dystrophy, wherein the sgRNA is capable of activating transcriptional and translational expressions of mouse utrophin; and DNA sequences of the sgRNA are as set forth in SEQ ID NOS: 1-20.

Based on a total technical conception, the present invention further provides a sgRNA for utrophin activation to treat Duchenne muscular dystrophy, wherein the sgRNA is capable of activating transcriptional and translational expressions of human utrophin; and DNA sequences of the sgRNA are as set forth in SEQ ID NOS: 21-39.

Based on a total technical conception, the present invention further provides use of the sgRNA as defined in preparation of a medicament for treating Duchenne muscular dystrophy.

Compared with the prior art, the present invention has the following advantages:
(1) the present invention provides the sgRNA for utrophin activation to treat DMD, sgRNA sequences capable of activating utrophin efficiently are derived by screening, and the mice sgRNAs are proved to be effective at the mouse cell level and the adult level; and human sgRNA sequences capable of activating utrophin efficiently have also been screened out, and these sgRNAs show great value in the future clinical translation of utrophin in place of dystrophin for the treatment of DMID.

### BRIEF DESCRIPTION OF THE DRAWINGS

To present the objects, technical solutions, and advantages of the embodiments of the present invention more clearly, the technical solutions of the embodiments of the present invention will be described clearly and completely with reference to the accompanying drawings in the embodiments of the present invention.

FIG. 1 shows the elementary diagram of the activation of a dCasMINI-VPR system;

FIG. 2 shows the schematic diagram of sgRNA locations in a mouse utrophin promoter region;

FIG. 3 shows the profile of a plasmid pX601-AAV-CMV-dCasMINI-VPR-EGFP-U6:BbsI-sgRNA;

FIG. 4 shows the profile of a plasmid pX601-AAV-spc5.12-dCasMINI-VPR-Cmyc-U6-M8-M12;

FIGS. 5A-5B show the screening results of effective sgRNAs in a promoter region of mouse utrophin in Experiment 1 of the present invention;

FIG. 6 shows the schematic diagram of construction of an integrated plasmid vector in Embodiment 1 of the present invention;

FIGS. 7A-7G show the evaluations of efficacy of utrophin activation treatment in mdx mice at an adult level in Experiment 2 of the present invention;

FIG. 8 shows the profile of a plasmid pX601-AAV-spc5.12-dCasMINI-VPR-Cmyc-U6-H1-H10;

FIGS. 9A-9B show the screening results of effective sgRNAs in a promoter region of human utrophin in Experiment 3 of the present invention; and

FIGS. 10A-10D show the results of utrophin activation in human skeletal muscle cells in Experiment 4 of the present invention.

### DETAILED DESCRIPTION OF THE EMBODIMENTS

The present invention will be further described below in conjunction with the specific preferred embodiments, but the protection scope of the present invention is not limited thereto. The materials and instrumentation used in the examples below are commercially available.

### Embodiment 1

For a sgRNA for utrophin activation to treat DMD, 20 sgRNAs M1 to M20 were designed 1000 bp upstream of the transcription start site of a mouse utrophin gene based on the PAM (TTR) sequence of a dCasMINI protein. Other DNA sequences can be found in Table 1 below.

| | | Gene sequence | | |
|---|---|---|---|---|
| No. | Name | No. | sgRNA sequence | PAM |
| 1 | M1 | SEQ ID NO: 1 | TGAAGTTTTTGACTTAATAAATG | TTTA |
| 2 | M2 | SEQ ID NO: 2 | GAGGGCAGGTCTTTTTCTGGACC | TTTG |
| 3 | M3 | SEQ ID NO: 3 | GGCCTGAGGAGTAGCAGAAGCTG | TTTG |
| 4 | M4 | SEQ ID NO: 4 | CTTACATGTCTTATCTCACCTGA | TTTG |
| 5 | M5 | SEQ ID NO: 5 | GGTTCTATTTGGCTTGTATGAAG | TTTA |
| 6 | M6 | SEQ ID NO: 6 | TAAAAAAGTCTTTTCCAAGACTT | TTTA |
| 7 | M7 | SEQ ID NO: 7 | TAAAGTCTCTGACTTAACAACTT | TTTA |
| 8 | M8 | SEQ ID NO: 8 | CTGGCTCATAAATTAACCCTTGA | TTTA |
| 9 | M9 | SEQ ID NO: 9 | GTTGCTATTTTTATTTCCTTCTC | TTTG |
| 10 | M10 | SEQ ID NO: 10 | TCAGGAAAACAGTAATGGGAACA | TTTA |
| 11 | M11 | SEQ ID NO: 11 | TTATACATTTCATTCTAACGTGT | TTTA |
| 12 | M12 | SEQ ID NO: 12 | CTTGGAAAAATAGAAAATAAATA | TTTG |
| 13 | M13 | SEQ ID NO: 13 | TGTTATGTATGCATTTTTATTAT | TTTG |
| 14 | M14 | SEQ ID NO: 14 | CGGGCTTAGCCGAGACCTATCAC | TTTA |
| 15 | M15 | SEQ ID NO: 15 | CAGAACTTGTTGGGAGTTTACGG | TTTG |
| 16 | M16 | SEQ ID NO: 16 | GGAAGCACGTCACACAGCCGAGG | TTTG |
| 17 | M17 | SEQ ID NO: 17 | GGTCTATCCTTCAAGGAGAGGAA | TTTG |
| 18 | M18 | SEQ ID NO: 18 | TCCGTGAAGCTCGGTGTCCATAA | TTTA |
| 19 | M19 | SEQ ID NO: 19 | GCAGCCTGTTTGTAATCCCAGCA | TTTG |
| 20 | M20 | SEQ ID NO: 20 | AATAAAGGGCACGGTGCGTGCGC | TTTG |

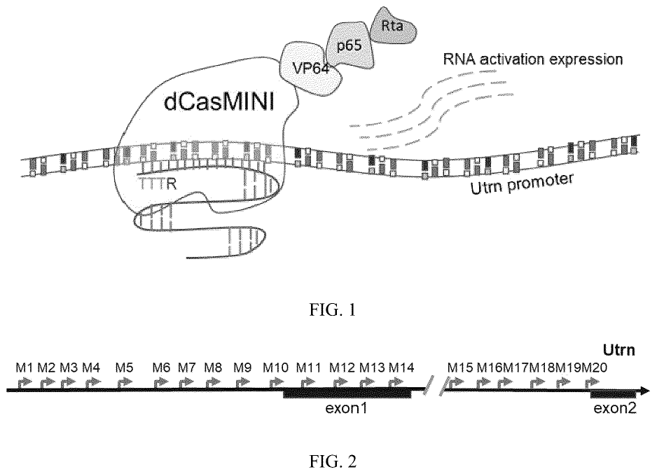

FIG. 1 shows the elementary diagram of the activation of a dCasMINI-VPR system, from which the targeting of a specific site by an inactivated dCasMINI protein-coupled activating element VPR (vp64/p65/Rta) under the guidance of sgRNA can be observed.

The dCasMINI contains 1584 base pairs, and Cas9 contains 4101 base pairs. dCasMINI is mini-Cas developed based on Cas12f, allowing for the construction of an integrated vector by means of the coupling activating element VPR. Cas protein, an RNA-guided endonuclease, is directed to a specific DNA sequence by means of the complementarity between an associated guide RNA (gRNA) and its target site. The Cas protein can almost point to any sequence with gRNA, as long as it is close to the short protospacer adjacent motif (PAM) site of the target. Cas9 variants (dCas9) that lack endonuclease activity but retain the ability to interact with DNA have been produced by mutation analysis. These inactivated dCas9 variants are then functionalized by means of effector domains such as transcriptional activation domains (ADs), enabling Cas9 to act as a cell programming tool at a transcriptional level. In a natural system, transcriptional initiation occurs by coordinating the recruitment of necessary mechanisms by means of many locally condensed transcription factors AD. The key functional domains of vp64/p65/Rta transcription factors are fused to enable a target gene to be activated at the transcriptional level under the guidance of gRNA in the dCasMINI-VPR system.

FIG. 2 shows the schematic diagram of sgRNA locations in a mouse utrophin promoter region;

### Embodiment 2

A vector was derived by cloning the sgRNA (M1 to M20) sequences from Embodiment 1 to a plasmid, respectively, which was then constructed into an AAV vector, with the steps as follows.
(1) A dCasMINI-VPR sequence was derived from a plasmid pHR-PGK-SV40_NLS-dCasMINI-V4-VPR-c-Myc_NLS-mCherry-WPRE (addgene: pSLQ9926).
(2) The dCasMINI-VPR sequence was subcloned into an AAV vector.
(3) dCasMINI and VPR elements were activated by using a CMV promoter, and then, a human U6 promoter was attached to the activated elements to activate the sgRNA and its crRNA. The attachment method could be found in Vazyme multi-fragment cloning kit (Vazyme: ClonExpress MultiS One Step Cloning Kit, Cat. No.: c113-01). A plasmid pX601-AAV-CMV-dCasMINI-VPR-EGFP-U6:BbsI-sgRNA (M1 to M20) was then obtained.

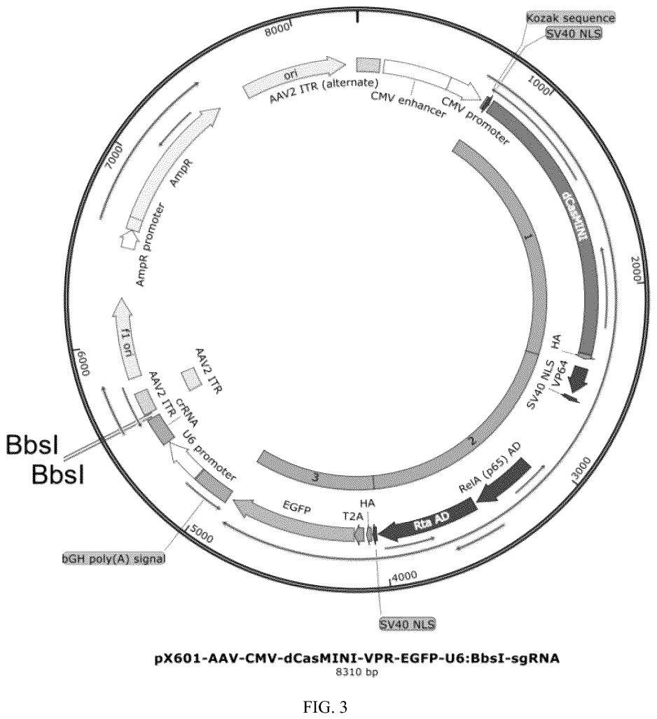

FIG. 3 shows the profile of the plasmid pX601-AAV-CMV-dCasMINI-VPR-EGFP-U6:BbsI-sgRNA.
(4) crRNA was digested with the restriction enzyme BbsI (NEB #R3539), followed by specific recognition of the site, agarose gel electrophoresis was carried out, and then the agarose gel was cut and extracted to obtain a linearized vector (for the step of gel extraction, refer to the instructions of EasyPure® Quick Gel Extraction Kit; Cat. No.: EG101-01). Double-stranded sgRNAs resulting from annealing were ligated with T4 ligase (for the step of ligation with T4 ligase, refer to the instructions of T4 DNA Ligase; Cat. No.: FL101-01).

In Embodiment 2, the plasmid pX601-AAV-CMV-dCasMINI-VPR-EGFP-U6:BbsI-M8 and the plasmid pX601-AAV-CMV-dCasMINI-VPR-EGFP-U6:BbsI-M12 were combined;
the plasmid pX601-AAV-CMV-dCasMINI-VPR-EGFP-U6:BbsI-M7 and the plasmid pX601-AAV-CMV-dCasMINI-VPR-EGFP-U6:BbsI-M8 were combined;
the plasmid pX601-AAV-CMV-dCasMINI-VPR-EGFP-U6:BbsI-M8 and the plasmid pX601-AAV-CMV-dCasMINI-VPR-EGFP-U6:BbsI-M10 were combined;
the plasmid pX601-AAV-CMV-dCasMINI-VPR-EGFP-U6:BbsI-M7 and the plasmid pX601-AAV-CMV-dCasMINI-VPR-EGFP-U6:BbsI-M10 were combined;
the plasmid pX601-AAV-CMV-dCasMINI-VPR-EGFP-U6:BbsI-M5 and the plasmid pX601-AAV-CMV-dCasMINI-VPR-EGFP-U6:BbsI-M8 were combined;
the plasmid pX601-AAV-CMV-dCasMINI-VPR-EGFP-U6:BbsI-M8 and the plasmid pX601-AAV-CMV-dCasMINI-VPR-EGFP-U6:BbsI-M13 were combined;
the plasmid pX601-AAV-CMV-dCasMINI-VPR-EGFP-U6:BbsI-M7, the plasmid pX601-AAV-CMV-dCasMINI-VPR-EGFP-U6:BbsI-M8 and the plasmid pX601-AAV-CMV-dCasMINI-VPR-EGFP-U6:BbsI-M13 were combined;
the plasmid pX601-AAV-CMV-dCasMINI-VPR-EGFP-U6:BbsI-M8, the plasmid pX601-AAV-CMV-dCasMINI-VPR-EGFP-U6:BbsI-M10 and the plasmid pX601-AAV-CMV-dCasMINI-VPR-EGFP-U6:BbsI-M12 were combined;
the plasmid pX601-AAV-CMV-dCasMINI-VPR-EGFP-U6:BbsI-M5, the plasmid pX601-AAV-CMV-dCasMINI-VPR-EGFP-U6:BbsI-M8 and the plasmid pX601-AAV-CMV-dCasMINI-VPR-EGFP-U6:BbsI-M10 were combined; and
the plasmid pX601-AAV-CMV-dCasMINI-VPR-EGFP-U6:BbsI-M8, the plasmid pX601-AAV-CMV-dCasMINI-VPR-EGFP-U6:BbsI-M10 and the plasmid pX601-AAV-CMV-dCasMINI-VPR-EGFP-U6:BbsI-M13 were combined, respectively.

Single sgRNA and the combinations of two or three sgRNAs were investigated in activation efficiency.

Experiment 1. Screening of Effective sgRNAs in Promoter Region of Mouse Utrophin:
(1) Neuro2a cells were seeded in a 12-well plate one day in advance. The cell density of 30% was preferred before transfection.
(2) Cell transfection was carried out using the vectors containing different sgRNAs from Embodiment 2 and the vectors 1 to 10 from Embodiment 3 with the Lipofectamine 2000 kit (Invitrogen, Cat. No.: 11668-027), respectively. The transfection was carried out at 1.5 μg of plasmid per well; 48 hours after transfection, the genomic RNAs of the cells were extracted with TRIzol (Invitrogen); and reverse transcription of cDNA was carried out using the Takara kit (PrimeScript™ RT reagent Kit with gDNA Eraser; Cat. No.: RR047A).
(3) QPCR was carried out with the TransGen kit (TransScript® Green One-Step qRT-PCR SuperMix; Cat. No.: AQ211-01) to verify the activation effect at the transcriptional level.

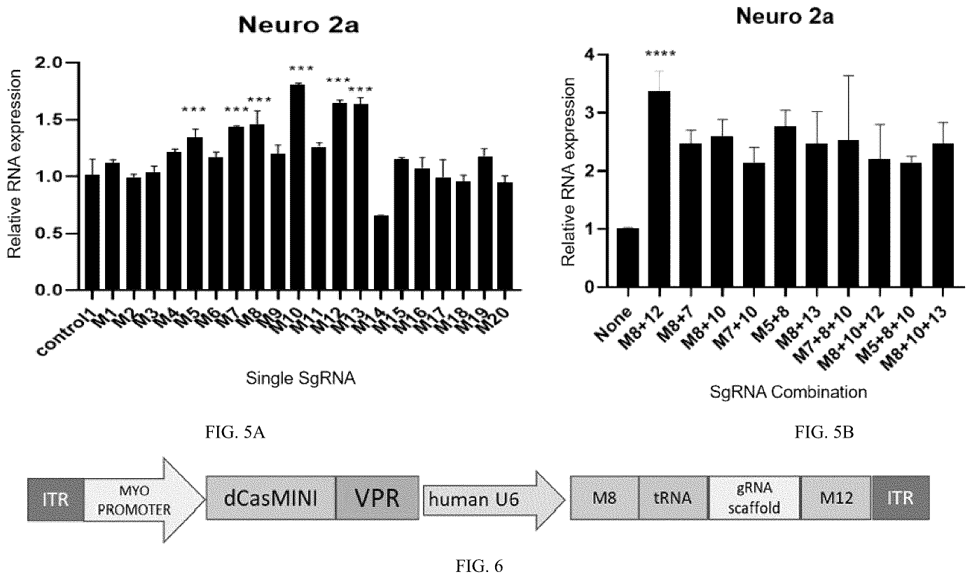

FIGS. 5A-5B show the screening results of effective sgRNAs in a promoter region of mouse utrophin. In FIGS. 5A-5B, FIG. 5A represents the schematic comparative diagram of qPCR results on the activation efficiency of single sgRNAs in a mouse Neuro2a cell line. FIG. 5B represents the schematic comparative diagram of the activation efficiency of the combinations of two and three sgRNAs. FIG. 6 shows the schematic diagram of construction of an integrated plasmid vector.

Based on the results in FIGS. 5A-5B, the plasmid dCasMINI-VPR-U6-sgRNA was constructed, and the sgRNAs with different sequences were linked to the vector. Subsequently, transfection was carried out in Neuro cells; 48 hours after transfection, RNA extraction was carried out for qPCR to verify the activation efficiency of single sgRNAs; the sgRNAs with the optima efficiency were selected and recombined; and the combinations of two and three sgRNAs were compared in activation efficiency. The combination with the highest activation efficiency was screened out, based on which an integrated vector was constructed. The optimal sgRNA sequence was M8: ctggctcataaattaacccttga and M12: cttggaaaaatagaaaataaata.

### Embodiment 3

A vector 1 in this embodiment was derived by: constructing M8 and M12 from Embodiment 1 into the same plasmid and then into an AAV vector to obtain a plasmid pX601-AAV-spc5.12-dCasMINI-VPR-Cmyc-U6-M8-M12. The construction method was the same as that in Embodiment 2.

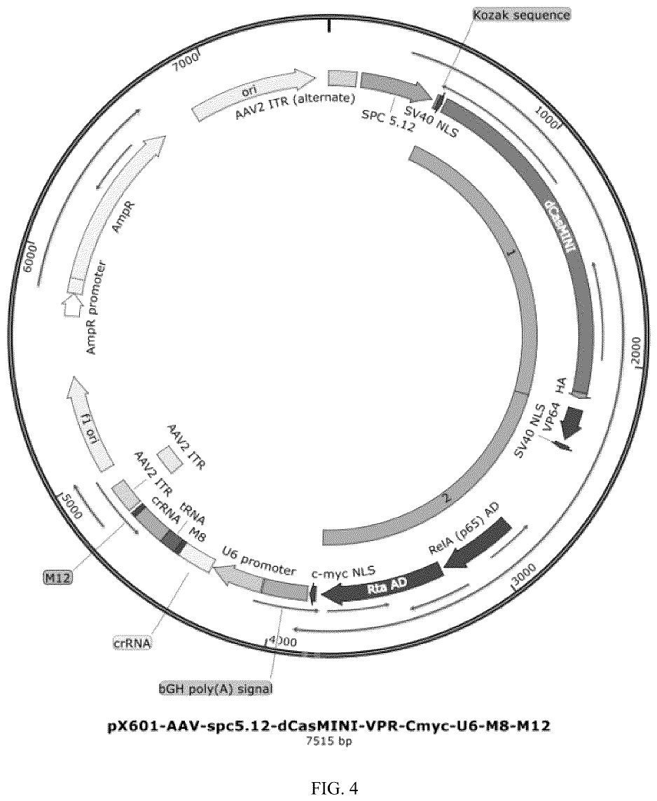

FIG. 4 shows the profile of the integrated plasmid pX601-AAV-spc5.12-dCasMINI-VPR-Cmyc-U6-M8-M12.

### Experiment 2. Evaluation of Efficacy of Utrophin Activation Treatment in Mdx Mice at Adult Level

The sgRNAs M8 and M12 with the best activation effects as verified in the mouse cell line Neuro2a were subjected to Myo virus serotype packaging (completed by PackGene Biotech). Mdx mice of 2 weeks old were injected through the tail vein at a dose of 3×1013 vg/kg, and 100 ul of normal saline was injected for the control group. The serum and muscle were collected at different timepoints for efficacy verification. Immunohistochemical antibodies (Dystrophin: abeam ab15277; utrophin: Leica NCL-DRP2; syntrophin: abeam ab11425) were prepared. The procedures of the immunohistochemical method and HE staining followed the conventional experimental procedures. The grip strength of front paws was evaluated by using the mouse paw grip strength gauge (Meilisai MC-RMG01). When the mice were lifted with their tails, their front paws were allowed to grip the steel grid connected to the device, respectively. Then, the mice were gently pulled through the steel grid, until the grip was released. The mice were tested 5 times successively, and the grip strength was calculated by averaging. The mouse exercise tolerance was evaluated with the mouse Rota rod (Meilisai MC-RMM-02) by comparing the time of falling from the Rota rod under uniform acceleration conditions. Western-blotting was carried out following the conventional experimental procedures.

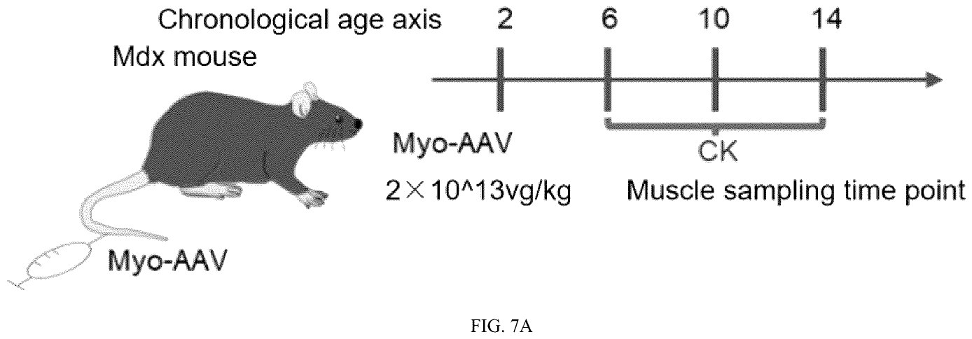

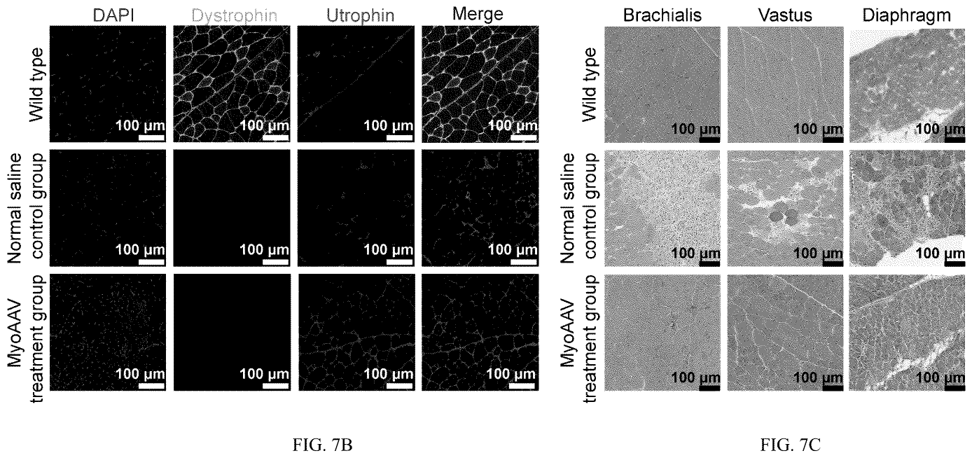

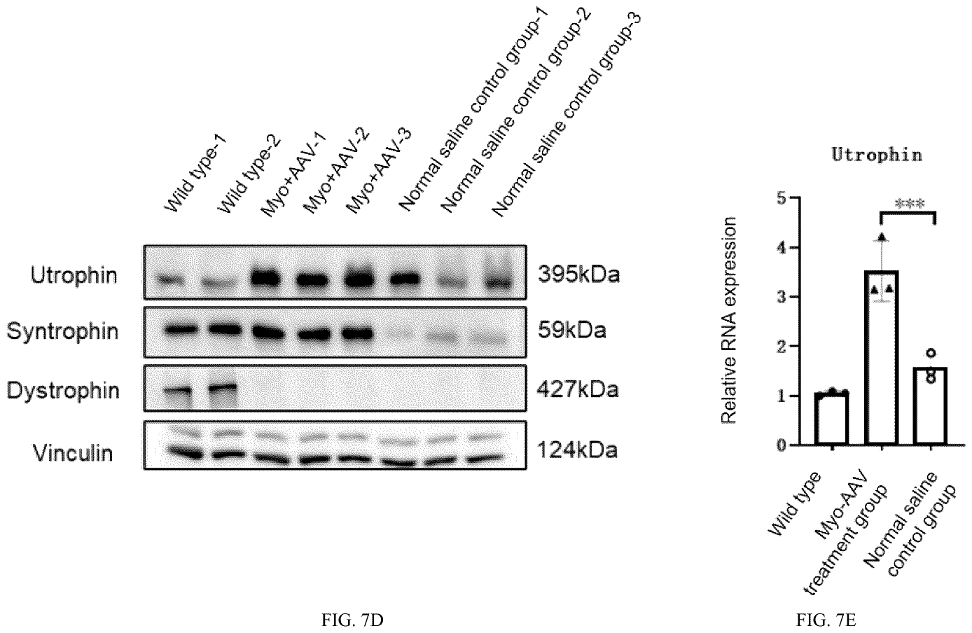

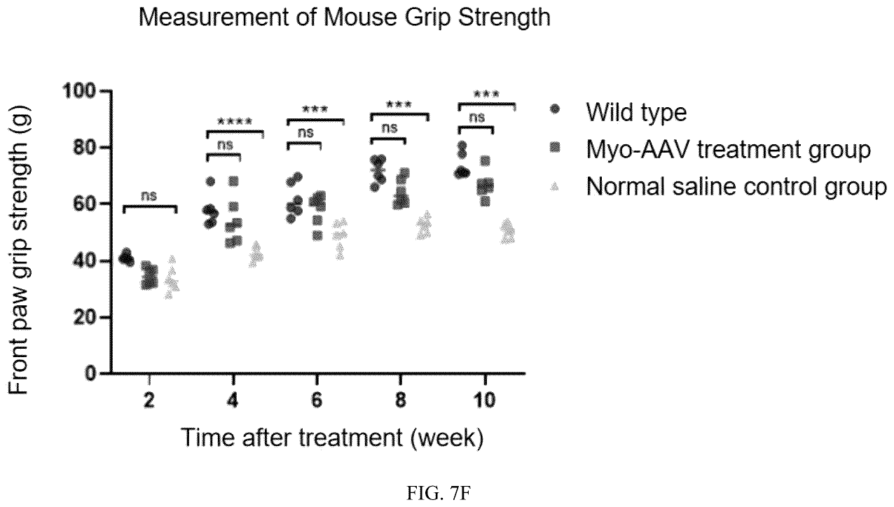

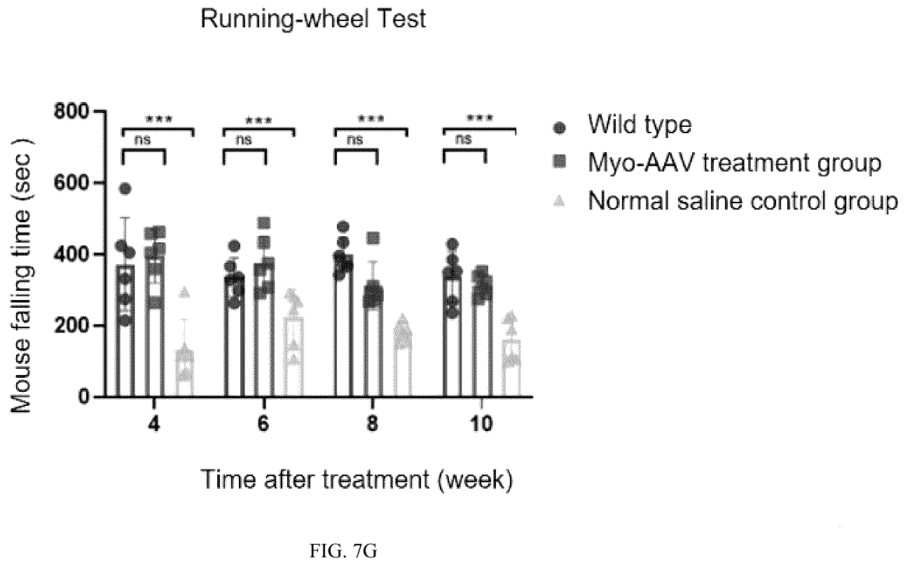

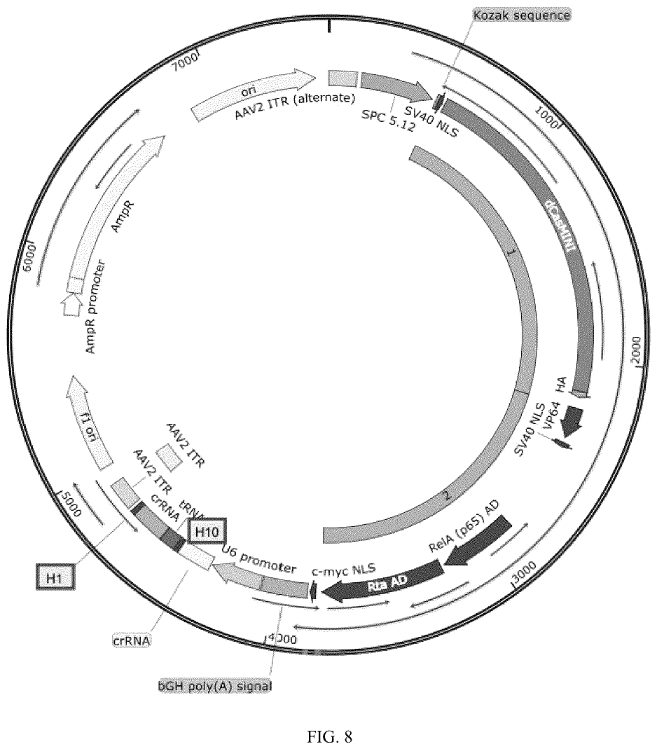

FIGS. 7A-7G show the evaluations of efficacy of utrophin activation treatment in mdx mice at the adult level. In FIGS. 7A-7G, FIG. 7A shows the age axis corresponding to the cycle of treatment for the mdx mice, depicting the schematic time diagram of Myo-AAV-mediated dCasMINI-VPR treatment in the mdx mice.

FIG. 7B shows the utrophin activation effect in muscle immunohistochemistry after treatment, in which the utrophin expression in the mouse muscle is shown significantly increased at Week 8 after treatment, and meanwhile, utrophin is mainly localized on the muscle cell membrane, which provides support for sustaining myolemma stability.

FIG. 7C shows the pathological diagram of HE in different muscles between the treatment group and the nontreatment group, in which the treatment group showed reduced inflammatory responses in different muscles and reduced in fibrotic area as compared to the nontreatment group.

FIG. 7D shows the Western-blotting results of verification of utrophin activation at the protein level after treatment. FIG. 7E shows the qPCR results of verification of utrophin activation at the RNA level after treatment. The results show a significant increase in utrophin expression at the protein and RNA levels in the AAV treatment group.

FIG. 7F shows the statistical results of mouse grip strength at different time points during the treatment. FIG. 7G shows the time for the mice to run on and fall from the running wheel at different time points during the treatment. The behavioral improvement of the mice at different weeks of age after treatment is shown. The grip strength of mice can effectively reflect the muscle strength. On Week 2 after treatment, there is no significant difference between the three groups; and on Week 4 after treatment, no significant difference can be seen between the AAV treatment group and the wild type group, but significant lack of muscle strength can be observed in the normal saline group. The running wheel test can effectively reflect the exercising tolerance of mice. When the mice fail to run on the wheel, they would fall from the rotating rod, and the time from the start of running to falling is recorded to reflect the exercising tolerance of the mice. At different weeks of age after treatment, the improved exercise performance can be seen from the mice in the AAV treatment group.

### Embodiment 4

For a sgRNA for utrophin activation to treat DMD, sgRNAs H1 to H19 for the PAM (TTTR) sequences of the dCasMINI protein were designed 1000 bp upstream of the transcription start site of a human utrophin gene. The DNA sequences can be found in Table 2 below.

| | | Gene | | |
|---|---|---|---|---|
| No. | Name | sequence No. | sgRNA sequence | PAM |
| 1 | H1 | SEQ ID NO: 21 | TCTCCCGGATCTTGTATATA | TTTA |
| 2 | H2 | SEQ ID NO: 22 | CATGTCTTATCTCACCTACT | TTTA |
| 3 | H3 | SEQ ID NO: 23 | AGGGTAAGATCACTATCAGA | TTTA |
| 4 | H4 | SEQ ID NO: 24 | TCTTTGCTTTACCCACAGAA | TTTA |
| 5 | H5 | SEQ ID NO: 25 | CTGGTTCTGTGGGTAAAGCA | TTTG |
| 6 | H6 | SEQ ID NO: 26 | GGGCTGTCAGGTTTGCTGGT | TTTA |
| 7 | H7 | SEQ ID NO: 27 | TTCATTCACTTCATTCATTC | TTTA |
| 8 | H8 | SEQ ID NO: 28 | ACAAGCATCAGTTTAGGGCT | TTTA |
| 9 | H9 | SEQ ID NO: 29 | TTTGGCTTATACGTAGAAAA | TTTA |
| 10 | H10 | SEQ ID NO: 30 | GGAATCTCCTACCTGCCCCC | TTTG |
| 11 | H11 | SEQ ID NO: 31 | TTATAGTGAAAGTTTCCCCC | TTTG |
| 12 | H12 | SEQ ID NO: 32 | AGGCCCCTAGTGGTTTAATC | TTTA |
| 13 | H13 | SEQ ID NO: 33 | CGCGGCAGTAAAGATTAAAC | TTTA |
| 14 | H14 | SEQ ID NO: 34 | CACATTTCATTGCTCGTGTG | TTTA |
| 15 | H15 | SEQ ID NO: 35 | GTCAACCTCTCAAGCTTGGA | TTTG |
| 16 | H16 | SEQ ID NO: 36 | TGTGTGCATATTGGAAAACA | TTTG |
| 17 | H17 | SEQ ID NO: 37 | TATTACAAGTAGTGTTTGCG | TTTG |
| 18 | H18 | SEQ ID NO: 38 | CATTATTTCTCTAGTGTCGT | TTTA |
| 19 | H19 | SEQ ID NO: 39 | CCCCTAGTTCCAACCGTATA | TTTA |

### Embodiment 5

According to the method in Embodiment 2, the sgRNA (H1 to H19) sequences from Embodiment 4 were respectively cloned to a plasmid and then constructed into an AAV vector to obtain plasmids pX601-AAV-CMV-dCasMINI-VPR-EGFP-U6:BbsI-sgRNA (H1 to H19).

In Embodiment 5, pX601-AAV-CMV-dCasMINI-VPR-EGFP-U6:BbsI-H10 and pX601-AAV-CMV-dCasMINI-VPR-EGFP-U6:BbsI-H6 were combined;
pX601-AAV-CMV-dCasMINI-VPR-EGFP-U6:BbsI-H10 and pX601-AAV-CMV-dCasMINI-VPR-EGFP-U6:BbsI-H1 were combined;
pX601-AAV-CMV-dCasMINI-VPR-EGFP-U6:BbsI-H10 and pX601-AAV-CMV-dCasMINI-VPR-EGFP-U6:BbsI-H4 were combined;
pX601-AAV-CMV-dCasMINI-VPR-EGFP-U6:BbsI-H10 and pX601-AAV-CMV-dCasMINI-VPR-EGFP-U6:BbsI-H12 were combined;
pX601-AAV-CMV-dCasMINI-VPR-EGFP-U6:BbsI-H10 and pX601-AAV-CMV-dCasMINI-VPR-EGFP-U6:BbsI-H13 were combined;
pX601-AAV-CMV-dCasMINI-VPR-EGFP-U6:BbsI-H10 and pX601-AAV-CMV-dCasMINI-VPR-EGFP-U6:BbsI-H11 were combined;
pX601-AAV-CMV-dCasMINI-VPR-EGFP-U6:BbsI-H1 and pX601-AAV-CMV-dCasMINI-VPR-EGFP-U6:BbsI-H6 were combined;
pX601-AAV-CMV-dCasMINI-VPR-EGFP-U6:BbsI-H4, pX601-AAV-CMV-dCasMINI-VPR-EGFP-U6:BbsI-H6 and pX601-AAV-CMV-dCasMINI-VPR-EGFP-U6:BbsI-H10 were combined;
pX601-AAV-CMV-dCasMINI-VPR-EGFP-U6:BbsI-H1, pX601-AAV-CMV-dCasMINI-VPR-EGFP-U6:BbsI-H6 and pX601-AAV-CMV-dCasMINI-VPR-EGFP-U6:BbsI-H10 were combined;
pX601-AAV-CMV-dCasMINI-VPR-EGFP-U6:BbsI-H6, pX601-AAV-CMV-dCasMINI-VPR-EGFP-U6:BbsI-H10 and pX601-AAV-CMV-dCasMINI-VPR-EGFP-U6:BbsI-H11 were combined;
pX601-AAV-CMV-dCasMINI-VPR-EGFP-U6:BbsI-H6, pX601-AAV-CMV-dCasMINI-VPR-EGFP-U6:BbsI-H10 and pX601-AAV-CMV-dCasMINI-VPR-EGFP-U6:BbsI-H12 were combined; and
pX601-AAV-CMV-dCasMINI-VPR-EGFP-U6:BbsI-H6, pX601-AAV-CMV-dCasMINI-VPR-EGFP-U6:BbsI-H10 and pX601-AAV-CMV-dCasMINI-VPR-EGFP-U6:BbsI-H13 were combined, respectively.

Single sgRNA and the combinations of two or three sgRNAs were investigated in activation efficiency.

Experiment 3. Screening of sgRNAs Capable of Efficiently Activating Promoter Region of Human Utrophin and their Related Combinations

The effectiveness of each sgRNA from Embodiment 5 at the cell level was verified following the method in Experiment 1.

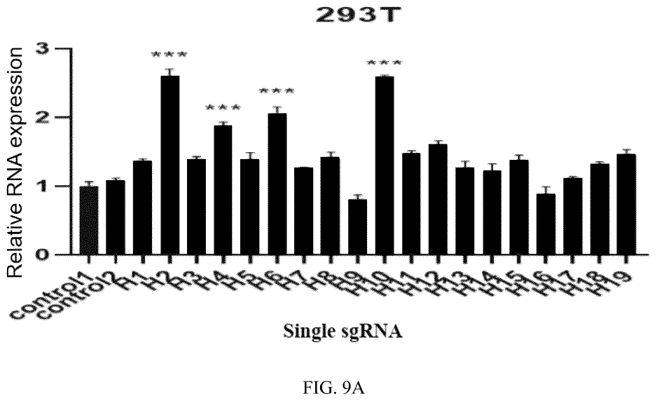

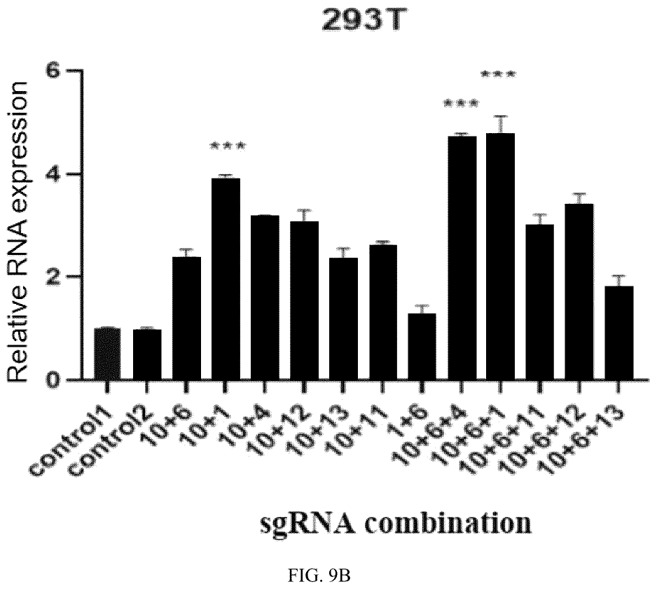

FIGS. 9A-9B show the screening results of effective sgRNAs in a promoter region of human utrophin. FIG. 9A represents the results of activation efficiency of single sgRNAs in 293T cells. FIG. 9B represents the combinations of sgRNAs with higher efficiency. Based on the results in FIGS. 9A-9B, it can be seen that the optimal combination of two sgRNAs as obtained after combining and screening is H1+H10.

### Embodiment 6

A vector in this embodiment was derived by: constructing H10 and H6 from Embodiment 5 into the same plasmid and then into an AAV vector to obtain pX601-AAV-spc5.12-dCasMINI-VPR-Cmyc-U6-H1-H10.

FIG. 8 shows the profile of the plasmid pX601-AAV-spc5.12-dCasMINI-VPR-Cmyc-U6-H1-H10.

### Experiment 4. Evaluation of Efficacy of Human Utrophin Activation Treatment

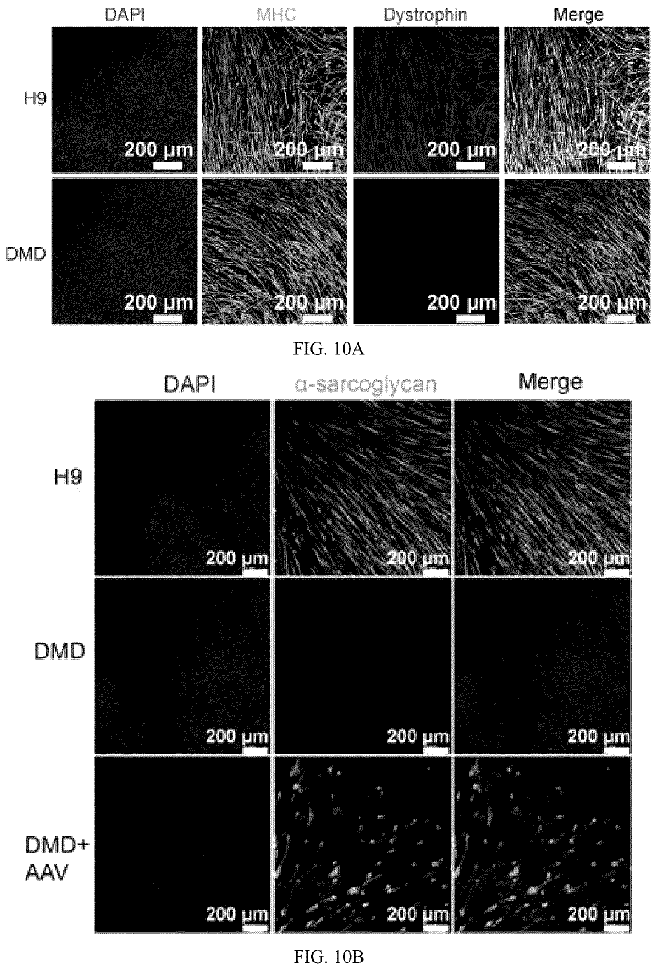

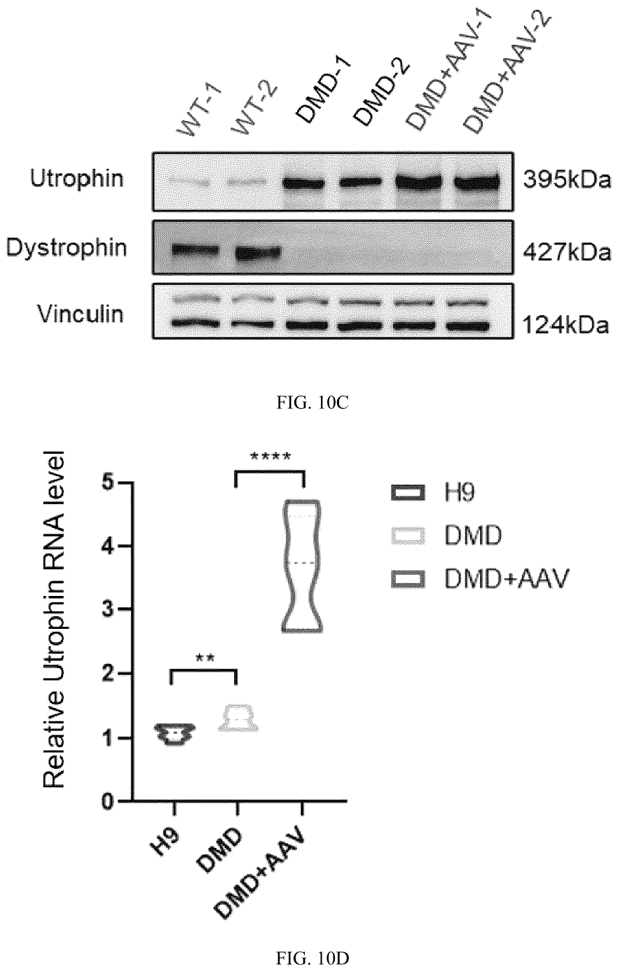

HEK293T cells were seeded in a 12-well plate one day in advance. The cell density of 30% was preferred before transfection. According to the method in Experiment 2, H1 and H10 were subjected to Myo virus serotype packaging, and the efficacy of human utrophin activation treatment was then evaluated. FIGS. 10A-10D show the utrophin activation results in human skeletal muscle cells, in which FIG. 10A shows that skeletal muscle cells differentiated from normal H9 cells form myotube-like cells and express dystrophin, and skeletal muscle cells derived from DMD patients do not express dystrophin. FIG. 10B shows that, after the utrophin activation treatment, the dystrophin-related protein α-sarcoglycan is also upregulated in the myotube-like cells of DMD patients, and the activation of surface utrophin promotes the restoration of muscle stability. FIGS. 10C-10D show the utrophin activation effects of utrophin detected at the protein and RNA levels after treatment.

Described above are merely preferred embodiments of the present invention, and are not intended to limit the present invention in any form. Although the present invention is disclosed as above in preferred embodiments, which are not intended to limit the present invention. Without departing from the spiritual essence and technical solutions of the present invention, any skilled person familiar with the art can make numerous possible variations and modifications to the technical solutions of the present invention, or amend these technical solutions into equivalent embodiments with equal changes, by using the methods and technical content disclosed above. Therefore, without departing from the contents of the technical solutions of the present invention, any simple alternations, equivalent substitutions, equivalent changes and modifications made to the embodiments above based on the technical essence of the present invention shall be construed as falling within the protection scope of the technical solutions of the present invention.

What is claimed is:

1. A sgRNA for a utrophin activation to treat a Duchenne muscular dystrophy, wherein the sgRNA is capable of activating transcriptional and translational expressions of a human utrophin; the sgRNA is a combination of H10 and H1, or a combination of the H10, H6 and the H1, or a combination of the H10, the H6 and H4;
wherein the DNA sequence encoding the H1 is as set forth in SEQ ID NO: 21;
the DNA sequence encoding the H4 is as set forth in SEQ ID NO: 24;
the DNA sequence encoding the H6 is as set forth in SEQ ID NO: 25; and
the DNA sequence encoding the H10 is as set forth in SEQ ID NO: 30.

2. A use of the sgRNA according to claim 1 in a preparation of a medicament for treating the Duchenne muscular dystrophy.

3. The use according to claim 2, wherein the combination of the H10 and the H1, or the combination of the H10, the H6 and the H1, or the combination of the H10, the H6 and the H4 is constructed in a same plasmid and then into an AAV vector for the utrophin activation.
